(19) United States
(12) Patent Application Publication
(10) Pub. No.: US 2005/0255525 A1
(43) Pub. Date: Nov. 17, 2005

(54) Safe method for isolation of prion protein and diagnosis of transmissible spongiform encephalopathies

(76) Inventor: Frank Owen Bastian, New Orleans, LA (US)

Correspondence Address: Frank Owen Bastian, 5207 Magazine Street, New Orleans, LA, 70115, US

(21) Appl. No.: 10/844,365

(22) Filed: May 13, 2004

(51) Int. Cl. C12Q 1/70; G01N 33/53

(52) U.S. Cl. 435/7.1

(57) ABSTRACT

Provided is a safe novel method for detecting Transmissible Spongiform encephalopathies (TSE). The method comprises: selecting a sample from a subject to determine whether the subject has transmissible spongiform encephalopathy; and detecting abnormal prion protein (Prpes) in the sample. The method detects PrP^{res} without Proteinase-K treatment by disrupting the sample in guanidine thiocyanate lysis solution followed by phenol purification of proteins, and demonstration of the abnormal prion isoform by Western blotting using monoclonal antibodies against prion protein structure. Guanidine salts effectively kill TSE infectivity providing a laboratory safe environment and stabilize biomolecules so TSE samples can be procured in the field and transported to the laboratory in guanidine lysis solution for processing at a later date. This method provides for rapid detection of the abnormal prion isoform diagnostic of TSE and results are easily interpretable based upon very different Western blot patterns for abnormal prion isoform versus the normal prion.

### CROSS-REFERENCE TO RELATED APPLICATIONS

U.S. Patent Documents

| | | | |
|---|---|---|---|
| 5808011 | September 1998 | Gawryl et al. | 530/416 |
| 6,150.172 | Nov. 21, 2000 | Schmerr et al. | |

### STATEMENT REGARDING FEDERALLY SPONSORED RESEARCH OR DEVELOPMENT

Funding for this project has been provided by National Institutes of Health grant # R01-NS044000.

### REFERENCE TO SEQUENCE LISTING, A TABLE, OR A COMPUTER PROGRAM LISTING COMPACT DISC APPENDIX

Not applicable

### BACKGROUND OF THE INVENTION

Throughout this application various publications are referenced, many in parenthesis. Full citations for each of these publications are provided at the end of the detailed description. The disclosures of each of these publications in their entireties are hereby incorporated by reference in this application.

The subject invention is directed generally to the detection of transmissible spongiform encephalopathies, and more particularly to a method of isolation of the hallmark prion protein that is diagnostic of TSE, which utilizes guanidine thiocyanate lyses solution to disrupt the tissue or body fluid samples followed by phenol purification of said proteins for sensitive, specific and reproducible demonstration of abnormal prion isoform by Western blotting that is representative of TSE in diseased tissues and not in controls.

A group of neurodegenerative diseases in humans and animals has been recognized as chronic infections of the central nervous system (CNS), and has been shown to be transmissible from one animal to another by inoculation (Bastian, 1991). The animal transmissible spongiform encephalopathies (TSE) include scrapie in sheep and goats, chronic wasting disease (CWD) in deer and elk, transmissible mink encephalopathy (TME) in farmed mink, and bovine spongiform encephalopathy (BSE) otherwise known as “mad cow disease”. Scrapie occurs worldwide and is endemic in English sheep. CWD is prevalent in the Western United States and involves 30% of the wild cervids (Spraker et al., 1997). BSE has so far been limited to Europe, Japan and Canada, although a case was recently discovered in Washington State (News Report). TSE in humans is referred to as Creutzfeldt-Jakob disease (CJD) which manifests as a rapidly progressive fatal dementia (Bastian, 1991). Although most CJD cases are sporadic (occur randomly with no apparent etiology), there are cases of CJD that have occurred following transplantation of dura or corneas derived from necropsy diseased tissues. In Europe, the outbreak of mad cow disease in humans, referred to as new variant CJD (Wills et al., 1996), demonstrated that TSE could be transmitted orally. There is also a great concern regarding possible transmission of CJD from one person to another by blood transfusion since TSE has been transmitted experimentally via blood (Houston et al., 2000) and over 200 professional blood donors have died of CJD (Vamvakas, 1999).

Since the pathology of TSE is characteristically spongiform degeneration of the neuropil of the brain, diagnosis may be made on examination of Hematoxylin and Eosin stained paraffin tissue sections, although there is often significant pathological variation between cases wherein some cases are difficult to interpret on routine histology (Bastian, 1991). Recognition of the neuropathology of TSE also requires the trained eye of a Neuropathologist or a pathologist with experience in examining TSE cases. Interpretation of the cases is enhanced by detection methods for a unique infection-related protein referred to as prion which accumulates in TSE tissues during infection (Ironside 1996). The abnormal prion protein is a modified cellular surface membrane glycoprotein (PrP) which is expressed normally and predominantly within the central nervous system (CNS), but with widespread distribution in tissues throughout the body (Prusiner, 1984). The pathophysiological mechanism of transformation of PrP^{c} to PrP^{res} is not known, but the hallmark for diagnosing TSE infection has become the demonstration of this protease-resistant prion isoform (PrP^{res}) in CNS and lymphoid tissues of suspect CJD cases (Ironside, 1996). Currently there is no workable preclinical diagnostic test for TSE based upon examination of urine, cerebrospinal fluid or peripheral blood. All diagnostic procedures for TSE can only reliably be done either on brain biopsy samples or necropsy specimens and those tests are currently based upon detection of the abnormal prion isoform.

### DESCRIPTION OF PRIOR ART

Screening of animals and human tissues for TSE is difficult and often unreliable in consideration of the current state of the art. Firstly, current methods for detection of PrP^{res} are complex in that they involve tissue dissociation, protease/detergent treatment, ultracentrifugation, followed by Western blot analysis (Madec et al., 1998; Baron & Biacabe, 2001). Secondly, these methods, based upon resistance of PrP^{res} to proteinase-K, are problematic in detecting prion isoforms since PrP^{c} of several mammalian species show intrinsic resistance to proteinase, K digestion (Buschmann et al., 1998). Furthermore, PrP^{res} may be protease-sensitive giving inconclusive results (Lasmezas et al., 1997). Therefore, these prion extraction methods require careful titration of amount of proteinase-K used (Barnard et al., 2000). Thirdly, another major problem with current methodology is that samples are potentially infectious at all stages of prion extraction (Madec et al, 1998; Bastian, 1991) so many diagnostic laboratories are not able or willing to process CJD brains. Fourthly, samples must be maintained fresh or frozen to be able to do Western blot assay. This makes obtaining optimal testable samples in the field difficult, near impossible. Fifthly, although PrP^{res} can be detected in formalin fixed tissues by immunohistochemistry (Ironside, 1996), frozen or fresh brain samples are more efficiently and reliably examined for presence of PrP^{res} by Western blot. Lack of consistency, low sensitivity and interpretation bias are major issues with immunohistochemistry. Sixthly, the need for optimum procurement of tissues has resulted in a significant number of inconclusive cases, especially in diagnostic studies of human tissues (P Gambetti, prion center). Lastly, investigation of the prion isoforms by direct comparison using current methodology is impossible since Pr^{c} is destroyed by proteinase-K while PrP^{res} is preserved (Prusiner, 1984). PrP^{c} has to isolated separately using immunoprecipitation while PrP^{res} is extracted as described supra. Therefore, we don't know the mechanism of transformation of one prion isoform to the other. The goal should be the development of a diagnostic test that is 100% effective without false positives while being sensitive enough to pick up all cases and that goal has not been achieved using current methodology.

### BRIEF SUMMARY OF INVENTION

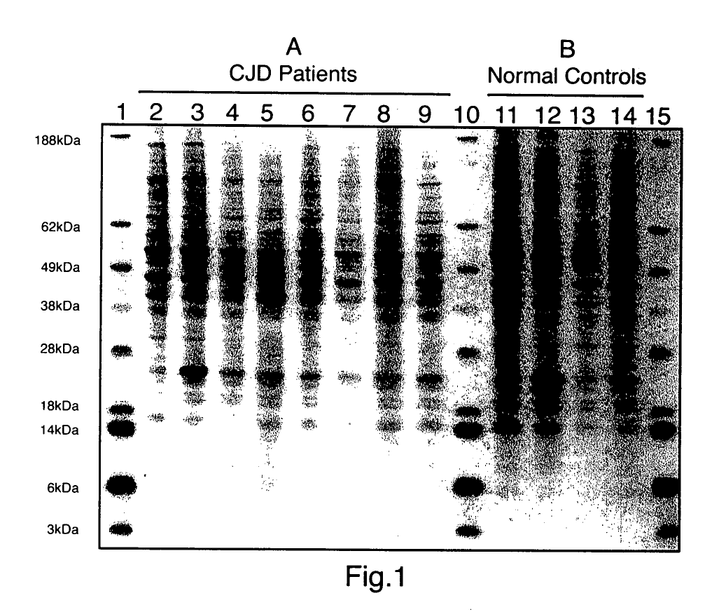

The method described here is a laboratory safe method for prion isolation using guanidine thiocyanate lyses of the sample followed by phenol/chloroform purification of the proteins. Following precipitation, the proteins are dissolved in SDS and submitted to sodium dodecyl suphate polyacrylamide gel electrophoresis (SDS PAGE). The gel is transferred to Immuno-blot membrane and Western blotted using monoclonal antibody against recombinant prion protein (we have found MAB 3F4 to work with human CJD cases but other monoclonals have worked with animal TSE cases suggesting that the nature of the antibody is not a limiting factor). Using this method, the typical normal prion immunoblot pattern was seen in all normal brains examined (15/15), whereas all CJD brains (18/18) showed a clearly distinct immunoblot pattern. Representative samples of CJD-infected and control samples are shown in FIG. 1. CJD diseased brains could be easily differentiated from normals by this method. We found that normal sheep brains (5) could be easily differentiated from scrapie infected sheep brains (5) using this method with monoclonal antibody specific for sheep prion (FIG. 5). This method is safe because the guanidine salts effectively kill the infectivity of the TSE samples, and, thus, this method can be used in both diagnostic and research laboratories. Since guanidine salts stabilize biomolecules for days to weeks, TSE samples can be procured in the field and added to the guanidine lyses buffer well before the assay is done making it applicable to any situation where there is problem in transfer. Diagnosis is available within 4-5 hours after the samples are submitted to the laboratory. The method also provides the prion isoforms in the same preparatory way allowing direct comparison research.

The advantages of this method over current methodology are: 1) This method for detection of PrP^{res} is simple, is not time consuming or technician labor intensive, and does not require special equipment; 2) This method is not based upon the use of proteinase-K to detect the prion isoforms and thus is not prone to the danger of false positives due to the eccentricities involved in using proteinase-K digestion (Buschmann et al., 1998). 3) Guanidine is efficient in killing the infectivity of the sample so that the methodology can be carried out in a routine laboratory situation without danger or concern to the technicians (Manuelidis, 1997). This procedure should alleviate the current fear by many diagnostic laboratories in their reluctance to handle processing of CJD cases; 4) procurement of tissues is always optimum since the sample is immediately dissolved in the guanidine lyses buffer and can be stored at room temperature, with the ability to obtain both nucleic acid and proteins several days later. We propose a kit for obtaining the samples at nominal cost, which would contain 20 vials each containing 800 microliter of guanidine thiocyanate lyses solution, 20 disposable scalpels and forceps for obtaining the sample, and instructions for transport; 5) This method negates the need for formalin fixation and thus samples can be optimally examined by Western blot rather than by error prone immunohistochemistry; 6) This modification should remove the occurrence of inconclusive cases, especially since we have found 100% correlation of positive test with CJD diagnosis and Western blot can be used in preference to error prone immunohistochemistry; 7) this method allows the ability to investigate normal or abnormal prion isoforms by direct comparison since proteinase-K is not used in the procedure and this method results in immunoprecipitation of both prion isoforms. The demonstration of both prion isoforms in the same preparation lends itself to automation wherein ratios of prion isoforms can be determined. In summary, a new method for prion extraction has been developed which is simple and can be applied to large numbers of samples. The method is safe in that guanidine salts effectively destroy infectivity so that this method can be adapted to most research laboratories.

### BRIEF DESCRIPTION OF VIEWS

FIG. 1. Coomassie blue stained acrylamide gel of eight CJD infected brains (A) and four normal human brains (B) where the proteins were extracted using our guanidine thiocyanate/phenol chloroform method. Lanes 1, 10, and 15, markers; Lanes 2 through 9, CJD-infected brains; Lanes 11 through 14, Normal Control brains. The preparation shows abundance of proteins retrievable from the phenol phase.

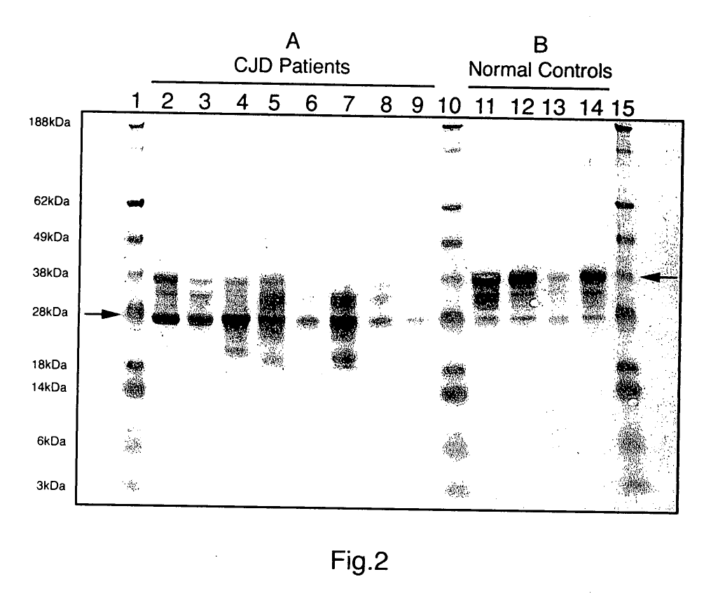

FIG. 2. Corresponding Western blot of preparation described in FIG. 1 showing that monoclonal antibody against recombinant prion protein (MAB 3F4) reacts with prion isoforms seen in eight CJD-infected brains (A) and four normal human brains (B). Lanes 1, 10, and 15, markers; Lanes 2 through 9, CJD-infected brains; Lanes 11 through 14, Normal Control brains. The most prominent bands seen in the normal controls are the diglycosylated bands at 38 kDa (right arrow) with monoglycosylated band at 33 kDa and unglycosylated band at 28 kDa. Lower bands are prominent in the CJD brains, particularly heavy at 27 kDa (left arrow) with even lower bands at 21 kDa and 18 kDa. The immunoblot patterns of prion isoforms in the CJD-infected brains are very different from the control brains.

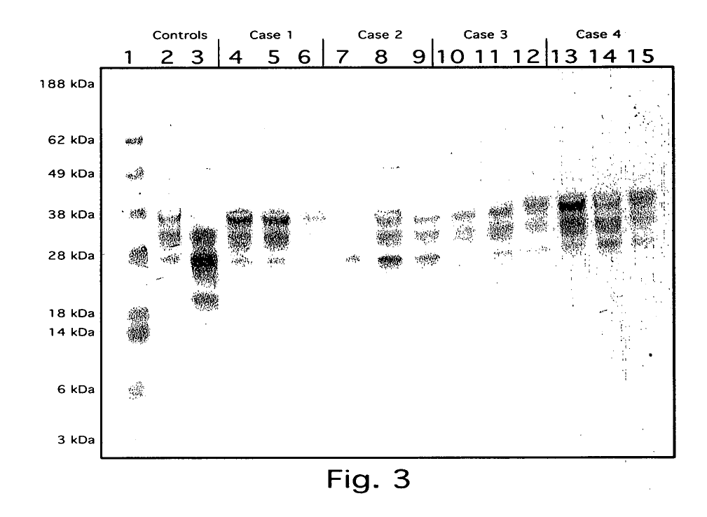

FIG. 3. Western blot of proteins derived from a series of Alzheimer human dementia brains where individual samples were taken from frontal cortex, basal ganglia or cerebellum using MAB 3F4. Lane 1, marker; Lane 2, control normal brain; Lane 3, CJD-infected brain; Lanes 4 through 6, Case #1-Alzheimer disease; Lanes 7 through 9, Case #2-abnormal pattern consistent with CJD; Lanes 10 through 12, Case #3-Alzheimer disease; Lanes 13 through 15, Case #4-Alzheimer disease. Abnormal prion isoform immunoblot patterns are seen in samples from case #2 indicative of CJD. The diagnosis of CJD was confirmed on histopathological review of case #2.

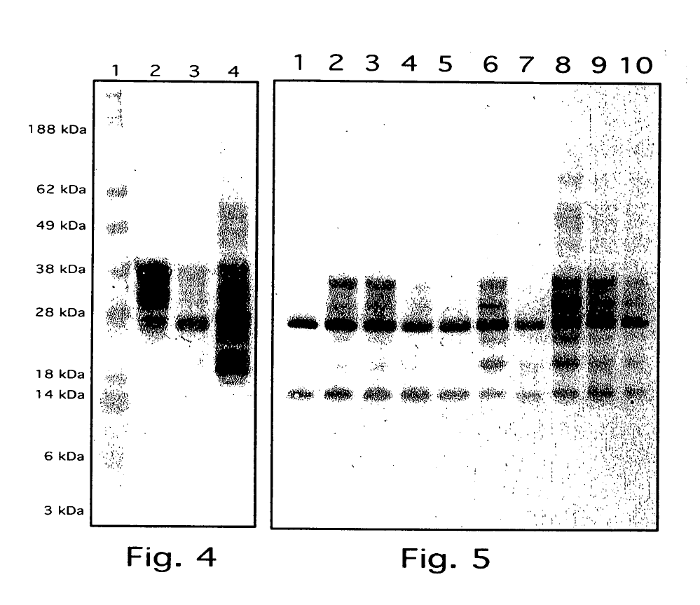

FIG. 4. Western blot of proteins extracted from an established case of Creutzfeldt-Jakob diseased brain where individual samples were taken from frontal cortex, basal ganglia or cerebellum using MAB 3F4. Lane 1, marker; Lane 2, frontal cortex; Lane 3, basal ganglia; Lane 4, cerebellum. Frontal cortex showed normal prion isoform pattern whereas basal ganglia and cerebellum showed abnormal prion protein consistent with CJD. The method clearly demonstrated absence of abnormal prion isoform in frontal cortex sample indicating that abnormal prion protein is irregularly deposited in established cases of CJD.

FIG. 5. Western blot of brains from normal and scrapie-infected sheep using this method and monoclonal 4B4 antibodies specific for sheep scrapie prion protein. Lane 1, marker; Lanes 2 through 6, normal sheep brain; Lanes 7 through 11, scrapie-infected sheep brains. Lower protein bands at 21 kDa indicative of abnormal prion protein were seen only in the scrapie-infected samples and therein demonstrated that the prion isoform pattern in scrapie sheep brain can be easily differentiated from normal sheep brain using this invention.

### DETAILED DESCRIPTION OF INVENTION

We have devised a safe method for purification and identification of abnormal prion protein isoform in TSE-infected samples. Although guanidine lyses is a component of many methods, especially for extraction of ribonucleic acids, this invention is the first demonstration of purification of abnormal prion protein combining guanidine lyses of the sample followed by phenol precipitation of the prion proteins. In this invention, a sample (100 mg of tissue for example) is placed in a 1.5 ml eppendorf tube to which is added 800 μl guanidine thiocyanate lyses buffer (4M GuSCN, 0.5% laurylsarcosine, 1 mM dithiothreitol, 0.3M NaCl in 0.1M tris buffer [pH 7.4]). The sample is homogenized with a sterile pestle and the guanidine lysed homogenate is incubated for 30 minutes at 37° C. followed by a 65° C. incubation for another 30 minutes. The preparation is then centrifuged for 20 min at 14,000 g, the pellet discarded, and the supernatant is placed in a new tube. 350 μl phenol/chloroform/isoamyl alcohol (25:24:1) is admixed with the supernatant, and the preparation is centrifuged for 5 min at 14,000 g at 4° C. The result is separation of an upper aqueous layer, an interphase layer and a lower phenol layer. The aqueous layer, which contains water soluble carbohydrates and nucleic acids is saved since it is suitable for future DNA or RNA extraction. The interphase, which contains denatured proteins, is discarded. The phenol layer is collected and proteins are precipitated from the phenol phase with 6 volumes isopropanol (Pusztai, 1966). The preparation is centrifuged for 5 min at 14,000 g at 4° C., the supernatant is discarded, and the alcohol is allowed to evaporate. The protein pellet is then dissolved in t % SDS buffer and electrophoresed on SDS-PAGE (4-12% Bis-Tris acrylamide gel)(Invitrogen). The proteins are transferred to Immuno-Blot PVDF membrane (Biorad) and immunoblotted with MAB 3F4 (Dako corporation) using Invitrogen's Western Breeze system which produces a finished blot in 4-5 hours.

Proteins precipitated from the phenol fraction when stained by Coomassie blue revealed multiple protein bands which appear to represent most of the sample proteins (FIG. 1). No significant protein bands are seen at 38 and 28 kDa on coomassie-stained preparations of CJD and normal brains as shown in FIG. 1. Western blot of the normal brain protein preparation revealed the characteristic PrP^{c} pattern typically produced by monoclonal prion antibodies (Baron & Biacabe, 2001) with bands at 38 kDa, 33-34 kDa, and 28 kDa (FIG. 2B). The CJD-infected brains immunoblotted with MAB 3F4 revealed a markedly different prion protein electrophoretic pattern with the major band at 28 kDa and lower bands at 18 kDa and 21 kDa (FIG. 2A). Protein preparations from CJD-infected brains shown in FIG. 2A revealed both prion isoforms, and the overall immunoblot patterns seen in the CJD patients were dramatically different as compared to the normal controls seen in FIG. 2B. Therefore, this invention produces prion isoform patterns that are diagnostic of this disease, which is clearly evident when matched with clinical history and neuropathology of the individual cases. We found that treatment of the preparations from both normal brain and CJD brain with a small amount of proteinase-K (5 μg/ml) for 2 minutes essentially removed both prion isoforms (data not shown) indicating that guanidine salts enhance activity of proteinase-K digestion.

Others have previously described extraction methods for detection of prion proteins without the need of proteolysis, but the results were ambiguous (Kang et al., 2003; Meyer et al., 1999). GdHCl and GdSCN solubilize PrPby disaggregation and may reveal previously inaccessible epitopes (Kang et al., 2003). We found that Western blot of protein extracted by the method revealed both prion isoforms without the need of proteinase K treatment. CJD cases showed distinct pattern differences compared to the normal prion immunoblot pattern. MAB 3F4 recognizes sequence 109-112 of human PrP so there may be unique changes due to the guanidine treatment that may expose epitopes at this site which allows differentiation of the abnormal prion isoform from the normal host prion immunoblot pattern. We have been successful in purifying both prion isoforms whether using guanidine thiocyanate or guanidine hydrochloride for lyses of the sample (data not shown for latter). Using the 4B4 monoclonal antibody specific for sheep prion protein, we found similar differences in immunoblots of prion proteins purified from normal sheep brain and scrapie-infected sheep brain (FIG. 5) wherein the scrapie abnormal prion could be easily identified in the scrapie-infected brains, but not seen in the normal sheep brains. The selection of the prion antibody is not a limiting factor for this invention since, in our hands, all prion monoclonal antibodies directed to the prion protein specific for each species of animal have worked well. Others have shown variable immunostaining with different monoclonal prion antibodies suggesting that one may have to be selective in choice of antibody to be used in our invention (Demart et al., 1999). In the future, we will continually test newer prion antibodies as they become available to determine the best fit for our detection method.

A major application of the invention was revealed when we found subtle changes in immunoblot pattern indicative of a hidden cases of TSE in review of a series of brains diagnosed as Alzheimer's disease. It has been reported that 13% of Alzheimer's disease brains have either associated CJD infection or are misdiagnosed and actually represent a case of CJD (Manuelidis, 1993). On study of twenty brains submitted from the Lousiana State University Medical center brain bank as Alzheimer's disease with our invention, we found several cases that had an immunoblot prion isoform pattern indicative of CJD. In FIG. 3 a representative group of Alzheimer cases from this series are shown wherein case number 2 has the immunoblot pattern of CJD. The diagnosis of CJD was confirmed on review of the neuropathology of that case which shows the reliability of the invention. Two other suspicious cases in this series (data not shown) also revealed spongiform encephalopathy on review of the histopathology. In addition, we showed that in an established case of CJD, that there is significant variation in abnormal prion deposition in samples from different sites in the brain (FIG. 4). In that case, the frontal cortex revealed lack of abnormal prion isoform whereas the basal ganglia and cerebellum revealed abundant abnormal prion isoform (FIG. 4). This variability in distribution of abnormal prion distibution in brains of CJD patient has been noted by others (Bell et al., 1997) and was the primary reason why we sampled several different sites on study of the Alzheimer cases. Therefore, our invention was not only able to detect CJD cases in a collection of brains misdiagnosed as Alzheimer's disease but also showed the importance of multiple sampling of such cases.

### EXAMPLES

This method provides significant advantages over currently used methods to extract prion proteins (Madec et al., 1998; Baron & Biacabe, 2001).

1 Provides a Safe Work Environment

Our guanidine method for extraction of prion protein from CJD brain tissues was developed primarily for laboratory safety reasons. Guanidine salts have been shown to effectively kill the transmissible agent of TSE (Manuelidis, 1997). The methodology of this invention differs markedly from other current methods where the sample is infectious at several stages of the purification procedure (Prusiner, 1984).

2 Provides Easy-To-Do Test Without Need for Special Equipment or Expert Training

This method is substantially less labor intensive than current methods used to purify prion proteins. Other currently used methods purify the prions by disrupting the tissues with proteases and detergents using repeated ultracentrifugations, using speeds requiring highly specialized expensive equipment available only in a research laboratory with a need for skilled technicians (Prusiner, 1984; Bastian, 1991). The method outlined in this invention is safe, requires minimal technician time, no specialized training or expertise and no specialized equipment.

3 Provides for Rapid Determination of Abnormal Prion in Sample

The evaluation is made within 4 hours after receipt of the sample: Briefly, the sample is incubated in the guanidine lyses buffer for one hour, treated with phenol from which the protein is extracted and run on Western blot.

4 Provides Abundant Protein from Sample for Other Assays.

As shown in FIG. 1, this method purifies abundant protein from the samples thereby allowing many experiments to be performed on the protein extracts.

5 Screening of Dementia Cases Suspect for CJD

In our hands, this method provides a reliable way to screen proteins extracted from brains of suspect CJD cases for presence of abnormal prion protein. All eight of CJD samples submitted from the prion center showed abnormal pattern consistent with prion disease. We have examined an additional ten CJD cases with the same results (data not shown). We found by using this invention that there is some variability in distribution of abnormal prion protein in CJD cases.

6 Screening of all Dementia Cases

We have had the opportunity to screen 20 cases at the LSU Dementia brain bank for abnormal prion proteins using this invention. Screening of Alzheimer cases in this fashion is important since 13% of cases are really cases of CJD, either in combination with Alzheimer's disease or representing a misdiagnosis. We found several cases in the LSU Alzheimer collection which show the immunoblot prion pattern consistent with CJD. In FIG. 3, case number 2 showed abnormal prion isoform pattern on Western blot and on further histopathological review revealed spongiform encephalopathy consistent with CJD.

7 Application to Neurosurgical Procedures

The rapid diagnosis possible using this invention allows significant advantages to protecting the neurosurgeon, the pathologist or the mortician. In the current atmosphere, the neurosurgeon operating on a dementia case for biopsy must compromise his surgery by using disposable instruments or risk contaminating his usual set of instruments, which may end up being used on several patients before a final diagnosis is obtained. Using our methodology, a diagnosis can be rendered within 4 hours after submission of the tissue to the laboratory which would allow the operating room to hold the surgeon's instruments until a decision can be made whether to dispose of certain of them and to perform more extensive cleanup of the operating room. Furthermore, the biopsy sample can be held for a few hours in formalin until a diagnosis is rendered. If a test based upon this invention is positive, the sample can then be processed in a separate histology laboratory so that other tissue samples are not contaminated.

8 Screening of Necropsy Tissues to be Used for Transplant

There are many instances of iatrogenic transmission of CJD through transplantation of dura or corneas derived from cadavers. At the time of obtaining the tissue from the cadaver, a small piece of the tissue can be lysed in the guanidine salt solution and submitted to the laboratory for detection of abnormal prion using this invention. The test results can be obtained before the sample homograft is placed in the tissue bank for distribution, thereby protecting the future patient from contamination by CJD through transplants.

9 Screening of Necropsy Tissues for Laboratory Safety

Similarly, if the pathologist must autopsy a dementia case, or a case without clinical history, a tissue sample can be taken from the brain and processed, while dissection of the organs can be delayed for one day. This will prevent contaminated tissues being processed along with routine specimens in the histology laboratory.

10 Screening of Postmortem Tissues for Embalming

Similarly, the mortician is often presented with a corpse where the clinical diagnosis is unknown or there is a history of dementia of unknown cause. A small brain sample can be obtained by trocar and examined by this method which would allow the mortician to proceed as usual or defer to use of special embalming methods or incineration so as to protect his facility from CJD contamination.

11 Field Testing of Animals for TSE

This method can also be easily applied to field studies where samples are obtained from suspect animals in a situation where tissues cannot be frozen and instead are preserved by fixation. Fixation with formalin does not allow for Western blotting or DNA studies and samples can only be studied by immunohistochemistry. This has presented a significant problem for the USDA since basing the entire assay procedures on immunohistochemical evaluation alone is known to give inconclusive results as well as show decreased sensitivity compared to other methods. Using our method, tissues are minced in the guanidine thiocyanate lyses buffer at the location where the sample is taken, which effectively kills the transmissible agent (Manuelidis, 1997) and stabilizes biomolecules for days until analysis in a laboratory. Western blotting could then give conclusive results in just a few hours following arrival to the laboratory. Immunoblot studies of sheep scrapie reveal presence of abnormal prion isoform which are not present in the normal sheep brain samples (FIG. 5).

12 Testing of Samples for TSE Front Animals Submitted for Taxidemmy

This method can easily and rapidly test whether samples submitted to a taxidermist for trophy preservation are infected with TSE. This would both protect the taxidermist as well as providing epidemiological evidence of spread of the disease into the local wild animal population.

13 Spot Checking of Meat Products

In the case of meat processing plants, a sample could be assayed by this invention while the meat is curing and long before meat is distributed to commercial outlets. Such testing would prevent a situation as occurred recently in Washington state where standard testing of a suspect case of BSE took over a week so that much of the meat from the BSE-infected Canadian cow had been distributed to the market place, and much had been consumed.

14 Spot Checking of Animal Products

Beef derived products are used in preparation of many commercial products including ice cream and lipstick. Fetal calf serum is widely used as an essential component of growth media for cell cultures. Samples of batches of serum or other beef products can be spot checked using this method to prevent distribution of a contaminated product. Similarly, food supplements derived from cattle or other animals can be checked. Of particular interest is the widespread use of elk velvet which should be screened since CWD is prevalent in the elk population.

15 Research Study of Prion Iosforms

Both prion isoforms can be compared by the same preparatory method. Direct comparison between the two prion isoforms has not been possible in the past since treatment with proteinase-K necessary to extract PrP^{res} destroys the PrP^{c} isoform (Bolton et al., 1987)). Current studies therefore are comparing prion protein isoforms each prepared very differently. Peptide mapping of these preparations from our study could lead to significant increase in our understanding of the nature of the prion isoforms including the relationship of the normal prion to the abnormal isoform and the mechanism of transformation of one isoform to the other. Since we have both prion isoforms in the same preparation using this invention, selective sequencing of the various fragments should allow us to better understand the nature of transformation of the normal prion isoform to the abnormal misfolded prion.

### REFERENCES CITED

1. Bastian F O. 1991. Review of theories of the transmissible agent, p. 49-64. In F O Bastian (ed), Creutzfeldt-Jakob disease and other transmissible spongiform encephalopathies. Mosby/Year Book Publishers, St. Louis, Mo.
2. Spraker T R, Miller M W, Willams E S, Getzy D M, Adrian W J, Schoonveld G G, Spowart R A, O'Rourke K I, Miller J M, Merz P A. Spongiform encephalopathy in free-ranging mule deer (Odocoileus hemionus), white-tailed deer (Odocoileus virginianus) and Rocky Mountain Elk (Cervus elaphus nelsoni) in northcentral Colorado. J Wildlife Dis 1997;33:1-6.
3. Will R G, Ironside J W, Zeidler M, Cousens S N, Estibeiro K, Alperovitch A, Poser S, Pocchiari M, Hofman A, Smith P G. A new variant of Creutzfeldt-Jakob disease in the UK. Lancet 1996;347:921-925.
4. Houston F, Foster J D, Chong A, Hunter N, Bostock C J. Transmission of BSE by blood transfusion in sheep. Lancet 2000;356:1771-1772.
5. Vamvakas E C. Risk of transmission of Creutzfeldt-Jakob disease by transfusion of blood, plasma, and plasma derivatives. J Clin Apheresis 1999;14:135-143.
6. Ironside J W. 1996. Neuropathological diagnosis of human prion disease: morphological studies, p. 35-38. In H F Baker and R M Ridley (eds.), Prion Diseases. Humana Press, Totowa, N.J.
7. Prusiner S B. 1984. Prions, p. 1245-1294. In B. N. Fields, D M Knipe, and P M Howley (eds.), Fundemental Virology. Lippincott-Raven Publishers, Philadelphia, Pa.
8. Madec J-Y, Groschup M H, Buschmann A, Belli P, Calavas D, Baron T. Sensitivity of the Western blot detection of prion protein PrPres in natural scrapie. J Virol Meth 1998;75:169-177.
9. Baron T, Biacabe A-B. Molecular analysis of the abnormal prion protein during co-infection of mice by bovine spongiform encephalopathy and scrapie agent. J Virol 2001;75:107-114.
10. Buschmann A, Kuczius T, Bodemer W, Groschup M H. Cellular prion proteins of mammalian species display an intrinsic partial proteinase K resistance. Biochem Biophys Res Commun 1998;253:693-702.
11. Lasmezas C I, Deslys J P, Robain O, Jaegly A, Beringue V, Peyrin J M, Fournier J G, Hauw J J, Rossier J, Dormond D. Transmission of the BSE agent to mice in the absence of detectable abnormal prion protein. Science 1997;275:402-405.
12. Barnard G, Helmick B, Madden S, Gilbourne C, Patel R. The measurement of prion protein in bovine brain tissue using differential extraction and DELFIA® as a diagnostic test for BSE. Luminescence 2000;15:357-362.
13. Manuelidis L. Decontamination of Creutzfeldt-Jakob disease and other transmissible agents. J Neurovirol 1997;3:62-65.
14. Pusztai A. Interactions of proteins with other polyelectrolytes in a two-phase system containing phenol and aqueous buffers at various pH values. Biochem J 1966;99:93-101.
15. Kang S-C, Li C, Wang C, Pan T, Liu T, Rubenstein R, Barnard G, Wong B-S, Sy M-S. Guanidine hydrochloride extraction and detection of prion proteins in mouse and hamster prion diseases by ELISA. J Pathol 2003;199:534-541.
16. Meyer R K, Oesch B, Fatzer R, Zurbriggen A, Vandevelde M. Detection of bovine spongiform encephalopathy-specific PrP(Sc) by treatment with heat and guanidine thiocyanate. J Virol 1999;73:9386-9392.
17. Demart S, Fournier J-G, Cremiinon C, Frobert Y, Lamoury F, Marce D, Lasmézas C, Dormont D, Grassi J, Deslys J-P. New insight into abnormal prion protein using monoclonal antibodies. Biochem Biophy Res. Comm. 1999;265:652-657.
18. Manuelidis E E, Manuelidis L. A transmissible Creutzfeldt-Jakob disease-like agent is prevalent in the human population. Proc Natl Acad Sci 1993;90:7724-7728.
19. Bell J E, Gentleman S M, Ironside J W, McCardle L, Lantos P L, Doey L, Lowe J, Fergusson J, Luthert P, McQuaid S, Allen I V. Prion protein immunocytochemistry-UK five centre consensus report. Neuropathol Appl Neurobiol 1997;23:26-35.
20. Bolton D C, Bendheim P E, Marmorstein A D, Potempska A. Isolation and structural studies of the intact scrapie agent protein. Archiv Biochem Biophys 1987;258:579-590.

1. A method of detecting transmissible spongiform encephalopathies, the method comprising:
Selecting a sample from a subject to determine whether the subject has a transmissible spongiform encephalopathy, and isolating abnormal prion proteins indicative of transmissible spongiform encephalopathy in the sample thereby detecting transmissible spongiform encephalopathy.

2. The method of claim 1 wherein the transmissible spongiform encephalopathy is Creutzfeldt-Jakob disease and the subject is human.

3. The method of claim 1 wherein the transmissible spongiform encephalopathy is bovine spongiform encephalopathy and the subject is bovine animal.

4. The method of claim 1 wherein the transmissible spongiform encephalopathy is scrapie and the subject is sheep.

5. The method of claim 1 wherein the transmissible spongiform encephalopathy is scrapie and the subject is mouse.

6. The method of claim 1 wherein the transmissible spongiform encephalopathy is chronic wasting disease and the subject is deer or elk (cervids).

7. The method of claim 1 wherein the transmissible spongiform encephalopathy is chronic wasting disease and the subject is farmed mink.

8. The method of claim 1 wherein the sample is a tissue sample.

9. The method of claim 8 wherein the tissue sample is brain.

10. The method of claim 8 wherein the tissue sample is spinal cord

11. The method of claim 8 wherein the tissue sample is extra-neural tissue.

12. The method of claim 11 wherein the extra-neural tissue is lymph node.

13. The method of claim 11 wherein the extra-neural tissue is tonsil.

14. The method of claim 1 wherein the sample is body fluid.

15. The method of claim 14 wherein the body fluid is cerebrospinal fluid

16. The method of claim 14 wherein the body fluid is peripheral blood

17. The method of claim 1 wherein the sample is secretion

17. The method of claim 16 wherein the sample is saliva

18. The method of claim 16 wherein the sample is seman

19. The method of claim 1 wherein the sample is excetion

20. The method of claim 19 wherein the sample is urine

21. The method of claim 19 wherein the sample is feces

22. The method of claim 1 wherein detecting abnormal prion proteins in the sample comprises lysis of the sample with guanidine salt solution and purification of prion proteins from the lysed sample preparation with phenol followed by Western blotting using prion-specific antibodies which demonstrate abnormal prion isoforms diagnostic of transmissible spongiform encephalopathy.

23. The method of claim 22 wherein the guanidine salt is guanidine thiocyanate

24. The method of claim 22 wherein the guanidine salt is guanidine hydrochloride

25. The method in claim 22 wherein prion-specific antibodies are monoclonal anti-prion antibodies.

26. The method in claim 22 wherein prion-specific antibodies are polyclonal anti-prion antibodies.

27. The method of claim 22 wherein the lysis of sample is in kit form

28. The method in claim 27 wherein the kit contains guanidine salt solution
